(19) United States
(12) Patent Application Publication
(10) Pub. No.: US 2024/0382394 A1
(43) Pub. Date: Nov. 21, 2024

(54) COMPOSITION COMPRISING AN OXIDATIVE DYE AND AMINE SALT

(71) Applicant: KAO CORPORATION, Tokyo (JP)

(72) Inventor: Celine MEULBROUCK, Darmstadt (DE)

(73) Assignee: KAO CORPORATION, Tokyo (JP)

(21) Appl. No.: 18/696,196

(22) PCT Filed: Sep. 29, 2022
(86) PCT No.: PCT/EP2022/077127

§ 371 Date: Mar. 27, 2024

(30) Foreign Application Priority Data
Sep. 30, 2021 (EP) 21200136.6

(51) Int. Cl. A61K 8/22; A61K 8/34; A61K 8/41; A61K 8/49; A61Q 5/10

(52) U.S. Cl. CPC A61K 8/22; A61K 8/347; A61K 8/41; A61K 8/411; A61K 8/4926; A61Q 5/10; A61K 2800/882

(57) ABSTRACT

A dyeing product for keratin fibers, including a composition C, containing a) 1,4-diamino-2-methoxymethyl-benzene, and/or a salt thereof. The dyeing product also includes an aqueous composition D having a pH in a range of 1 to 6 containing b) an alkylamine salt or alkanolamine salt and g) hydrogen peroxide. The composition C and the aqueous composition D are kept separate until directly prior to use onto the keratin fibers.

### FIELD OF THE INVENTION

The present invention relates to a dyeing product and dyeing method for keratin fibers with 1,4-diamino-2-methoxymethyl-benzene in a first composition and an alkylamine or alkanolamine in a second oxidizing composition.

### BACKGROUND OF THE INVENTION

Oxidative dyes possess superior dyeing ability and grey coverage, but in some cases their wash fastness is impaired. Depending on the history of the dyed keratin fibers, their kinetics of fading over shampooing cycles differ.

This may result in undesired color changes of the customer's keratin fibers, especially hair, leading to dissatisfaction and shorter periods between hair dyeing sessions.

EP3427720 discloses amine salts in oxidizing compositions, but is silent on the nature of dyes and technical effects of the present invention.

### SUMMARY OF THE INVENTION

Therefore, the first object of the present invention is a dyeing product for keratin fibers, preferably human keratin fibers, more preferably human hair, comprising:
a composition C comprising
a) 1,4-diamino-2-methoxymethyl-benzene, and/or its salt(s),
an aqueous composition D having a pH in the range of 1 to 6 comprising
b) one or more alkylamine salt(s) or alkanolamine salt(s) according to the following general structure

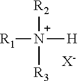

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and X— is an anion, and
g) hydrogen peroxide.

The second object of the present invention is
a method for dyeing keratin fibers, preferably human keratin fibers, more preferably human hair, comprising the steps of:
x) providing composition C as defined above and mixing it with an composition D as defined above, to yield a ready-to-use composition having a pH in the range of 7 to 12,
xi) applying the ready-to-use composition onto keratin fibers and leaving it for a time period in the range of 1 min to 60 min,
xii) optionally rinsing-off the keratin fibers and optionally drying the keratin fibers.

### DETAILED DESCRIPTION OF THE INVENTION

Inventors of the present invention have unexpectedly found out that the dyeing product according to claim 1 has superior wash fastness, in particular on pre-damaged hair. In addition, it delivers a superior dyeing intensity.

### Dyeing Product

The present invention is directed to a dyeing product for keratin fibers, preferably human keratin fibers, more preferably human hair, comprising:
a composition C comprising
a) 1,4-diamino-2-methoxymethyl-benzene, and/or its salt(s),
an aqueous composition D having a pH in the range of 1 to 6 comprising
b) one or more alkylamine salt(s) or alkanolamine salt(s) according to the following general structure

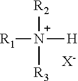

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and X— is an anion, and
g) hydrogen peroxide.

Suitable salts of compound(s) according to group a) is/are sulfate salt, hydrochloride salt, hydrobromide salt, nitrate salt, phosphate salt, hydrogen phosphate salt, dihydrogen phosphate salt, methosulfate salt, citrate salt, succinate salt, tartrate salt, lactate salt, tosylate salt, benzenesulfonate salt, acetate salt, and/or their mixtures, preferably it/they is/are a sulfate salt and/or a hydrochloride salt, and/or their mixtures, more preferably it is a sulfate salt, form the viewpoint of speed of dissolution.

It is preferred from the viewpoint of dyeing intensity that the total concentration of compound(s) according to group a) in composition C is 0.01% by weight or more, preferably in the 0.02% by weight or more, further more preferably 0.05% by weight or more, still more preferably 0.1% by weight or more, calculated to the total weight of composition C.

It is preferred from the viewpoint of cosmetic safety that the total concentration of compound(s) according to group a) in composition C is 65% by weight or less, preferably 50% by weight or less, further more preferably 35% by weight or less, still further more preferably 30% by weight or less, still further more preferably 20% by weight, calculated to the total weight of composition C.

For attaining the above-mentioned effects, it is preferred that the total concentration of compound(s) according to group a) in composition C is in the range of 0.01% to 65% by weight, preferably in the range of 0.02% to 50% by weight, more preferably in the range of 0.05% to 35% by weight, still more preferably in the range of 0.1% to 30% by weight, still further more preferably in the range of 0.1% to 20% by weight, calculated to the total weight of composition C.

Optionally, composition C may comprise one or more oxidative dye precursor or oxidative dye coupler different from group a), preferably 5-amino-2-methylphenol, 2-methyl-5-hydroxyethylaminophenol, 2,4,-diaminophenoxyethanol, 2-amino-4-hydroxyethylaminoanisol, 2-methyl-5-amino-6-chlorphenol, 1,3-bis-(2,4-diaminophenoxy)-propane, 2-bis(2-hydroxyethyl)-aminotoluene, 2-amino-5-methylphenol, resorcinol, 2-methylresorcinol, 4-chlororesorcinol, 2-amino-4-chlorophenol, 5-amino-4-methoxy-2-methylphenol, 2-aminophenol, 3-amino-phenol, 1-methyl-2-hydroxy-4-aminobenzene, 3-N,N-dimethyl aminophenol, 2,6-dihydroxy-3,5-dimethoxypyridine, 5-amino-3-methylphenol, 6-amino-3-methylphenol, 2,4-diaminophenoxyethanol, 1,3-diamino-benzene, 1-amino-3-(2′-hy-droxyethylamino) benzene, 1-amino-3-[bis(2′-hydroxy-ethyl)amino]benzene, α-naphthol, 4,6-dichlororesorcinol, 1,3-diamino-toluene, 4-hydroxy-1,2-methylenedioxy benzene, 1,5-dihydroxy naphthalene, 1,6-dihydroxy naphthalene, 1,7-dihydroxy naphthalene, 2,7-dihydroxy naphthalene, 1-hydroxy-2-methyl naphthalene, 4-hydroxy-1,2-methyldioxy benzene,2,4-diamino-3-chlorophenol, 5-amino-2-methoxyphenol and/or 1-methoxy-2-amino-4-(2′-hydroxyethyl amino)-benzene, 2,4,5,6-tetraaminopyrimidine, 2,5,6-triamino-5-pyrimidinol, and/or their salt(s), and/or their salt(s), and/or their mixtures, preferably one or more compound(s) according to group b) is/are selected from 4-chlororesorcinol, 2-methylresorcinol, 1,3-bis-(2,4-diaminophenoxy)-propane, 2-methyl-5-amino-6-chlorphenol, 2-amino-3-hydroxypyridine, 5-amino-6-chloro-o-cresol, 3-amino-2,4-dichlorophenol, and/or their salt(s), and/or their mixtures, more preferably 1,3-bis-(2,4-diamino-phenoxy)-propane, 2,4-diaminophenoxyethanol, 2-amino-4-hydroxyethylaminoanisole, 5-amino-6-chloro-o-cresol, 3-amino-2,4-dichlorophenol, and/or their salt(s), and/or their mixtures.

It is preferred from the viewpoint of dyeing intensity that the total concentration of one or more oxidative dye precursor or oxidative dye coupler different from group a) is 0.01% by weight or more, preferably in the 0.05% by weight or more, further more preferably 0.08% by weight or more, calculated to the total weight of composition C.

It is preferred from the viewpoint of cosmetic safety that the total concentration of one or more oxidative dye precursor or oxidative dye coupler different from group a) is 20% by weight or less, preferably 15% by weight or less, further more preferably 10% by weight or less, still further more preferably 5% by weight or less, still further more preferably 3.5% by weight or less, calculated to the total weight of composition C.

For attaining the above-mentioned effects, it is preferred that the total concentration of one or more oxidative dye precursor or oxidative dye coupler different from group a) is in the range of 0.01% to 20% by weight, preferably in the range of 0.05% to 15% by weight, more preferably in the range of 0.08% to 10% by weight, still more preferably in the range of 0.08% to 5% by weight, still further more preferably in the range of 0.08% to 3.5% by weight, calculated to the total weight of composition C.

It is preferred from the viewpoint of user convenience that composition C comprise one or more alkalizing agent(s), preferably one or more inorganic or organic alkalizing agent(s), and/or their mixtures.

Preferably, composition C comprise one or more inorganic alkalizing agent(s), more preferably they are metasilicates, disilicates, carbonates, bicarbonates, and/or phosphates, and/or their metal salts such as alkali or earth alkaline salts, and/or their mixtures, preferably it is sodium metasilicate, trisodium phosphate and/or tripotassium phosphate, and/or their mixtures.

In addition, or alternatively, composition C may comprise one or more organic alkalizing agent(s) such as ammonia and/or its salt(s), one or more organic alkyl and/or alkanol amines according to the following general structure:

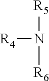

wherein R₄, R₅, and R₆ are independently selected from H, linear C₁-C₆ alkyl which may be substituted with one hydroxyl group, or branched C₃-C₁₂ alkyl or alkanol, wherein at least one of R₄, R₅, or R₆ is different from H, and/or their mixtures.

Preferably, one or more organic alkalizing agent(s) are selected from alkyl and/or alkanolamine(s), more preferably they/it is selected from monoethanolamine, diethanolamine, monoethanol methylamine, monoethanol dimethylamine, diethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine, trimethylamine, triethylamine, 2-amino-2-methylpropanol, tris-(hydroxymethyl)-aminomethane, and/or their mixtures, from the viewpoint of providing alkalinity and cosmetic safety as well as their low odor.

The most preferred alkalizing agent(s) is/are selected from monoethanolamine, 2-amino-2-methylpropanol, tris-(hydroxymethyl)-aminomethane, ammonium hydroxide, and/or their mixtures, from the viewpoint of providing alkalinity and cosmetic safety.

It is preferred from the viewpoint of dyeing intensity that the total concentration of alkalizing agents, preferably of inorganic or organic alkalizing agents, is 0.1% by weight or more, more preferably 0.2% by weight or more, further more preferably 0.5% by weight or more, calculated to the total weight of composition C.

It is preferred from the viewpoint of cosmetic safety that the total concentration of alkalizing agents, preferably of inorganic or organic alkalizing agents, is 40% by weight or less, more preferably 30% by weight or less, further more preferably 20% by weight or less, calculated to the total weight of composition C.

For attaining the above-mentioned effects, it is preferred that the total concentration of alkalizing agents, preferably of inorganic or organic alkalizing agents, is in the range 0.1% to 40% by weight, preferably in the range of 0.2% to 30% by weight, more preferably in the range of 0.5% to 20% by weight, calculated to the total weight of composition C.

### Direct Dyes

Optionally, composition C may comprise one or more direct dye(s).

Suitably, one or more direct dye(s) may be HC Blue 18, HC Red 18, HC Yellow 16, Disperse Black 9, Acid Yellow 1, 2-amino-6-chloro-4-nitrophenol, and/or their salt(s), and/or their mixtures.

Suitably, the total concentration of direct dye(s) is 0.01% by weight or more, preferably 0.05% by weight or more, more preferably 0.1% by weight or more, calculated to the total weight of composition C.

Suitably, the total concentration of direct dyes is 5% by weight or less, preferably 2.5% by weight or less, more preferably 1.5% by weight or less, calculated to the total weight of composition C.

Suitably, the total concentration of direct dyes is in the range of 0.01% to 5% by weight is in the range of 0.05% to 2.5% by weight, more preferably in the range of 0.1% to 1.5% by weight, calculated to the total weight of composition C.

### Product Forms of Composition C

Composition C of the present invention may be realized in various cosmetic product forms.

### Aqueous Composition

It is preferred from the viewpoint of user convenience that composition C of the present invention is an aqueous composition.

The term ‘aqueous’ denotes a composition C that comprises a majority of water, i.e., composition C preferably comprises water at a total concentration of 50% by weight or more, further more preferably at 60% by weight or more, still more preferably at 70% by weight or more, still further more preferably at 80% by weight or more, calculated to the total weight of composition C, from the viewpoint of achieving a cosmetically acceptable composition.

It is further preferred from the viewpoint of user convenience that composition C comprises water at a total concentration of 98% by weight or less, more preferably at 95% by weight or less, further more preferably at 92% by weight or less, calculated to the total weight of composition C.

For achieving the above-mentioned effects, it is preferred that the total concentration of water in composition C is in the range of 50% to 98% by weight, more preferably in the range of 60% to 95% by weight, further more preferably in the range of 70% to 92% by weight, still more preferably in the range of 80% to 92% by weight, calculated to the total weight of each of the composition C.

It is preferred from the viewpoint of dyeing performance that the pH of composition C is 7 or more, more preferably the pH is 8 or more, further more preferably the pH is 8 or more, still further more preferably the pH is 9 or more.

It is preferred from the viewpoint of hair damage and dyeing performance that the pH of composition C is 12 or less, more preferably the pH is 11.5 or less, still more preferably the pH is 10.5 or less.

For attaining the above mentioned effects, it is preferred that the pH of composition C is in the range of 7 to 12, preferably in the range of 8 to 11.5, further more preferably in the range of 9 to 11.5, further more preferably in the range of 9 to 10.5.

Preferably, the pH is measured with a glass electrode at 25° C. under atmospheric pressure.

### Powder Composition—compound(s) According to Group c)

It is preferred from the viewpoint of composition stability and convenience of use that compositions C comprise one or more pulverulent excipient as compound(s) according to group c).

The term ‘excipient’ denotes a compound, which may act as filling material and dispersant for the other compounds of composition C and does not react with the dyes or the alkalizing agent, and, thus, confer the powder a high degree of storage stability over an extended period of time.

Composition C of the present invention may comprise an organic and/or an inorganic pulverulent excipient in which dyes are dispersed.

Suitable organic and/or an inorganic pulverulent excipients are, for example, diatomaceous earth, kaolin, bentonite, starch especially corn, tapioca, rice, wheat and potato, nylon powder, montmorillonit, gypsum, sawdust and perlite, more preferably it is corn starch, from the viewpoint of stability.

Preferably, the total concentration of compound(s) according group c), more preferably the total concentration of organic and/or an inorganic pulverulent excipients, is 10% by weight or more, more preferably 15% by weight or more, further more preferably 20% by weight or more, still further more preferably 25% by weight or more, even further more preferably 30% by weight or more, even more preferably 35% by weight or more, calculated to the total weight of composition C, from the viewpoint of achieving good dispersability of the direct dyes in the powder and quick dissolution of the powder.

Preferably, the total concentration of compound(s) according group c), more preferably the total concentration of organic and/or an inorganic pulverulent excipients, is 98% by weight or less, more preferably 95% by weight or less, further more preferably 90% by weight or less, calculated to the total weight of composition C, from the viewpoint of achieving good dispersability of the dyes in the powder and formulation freedom.

For attaining the above mentioned effects, it is preferred that the the total concentration of compound(s) according group c), more preferably the total concentration of organic and/or an inorganic pulverulent excipients, is in the range of 10% to 98% by weight, more preferably in the range of 15% to 95% by weight, further more preferably in the range of 20% to 90% by weight, still further more preferably in the range of 25% to 90% by weight, still further more preferably in the range of 30% to 90% by weight, even more preferably in the range of 55% to 90% by weight, calculated to the total weight of composition C.

It is further preferred from the viewpoint of stability that the total concentration of water in composition C is 10% by weight or less, preferably 5% by weight or less, more preferably 1% by weight or less, still more preferably 0.1% by weight or less, still more preferably composition C is anhydrous.

The term ‘anhydrous’ denotes a composition that is free of added water. However, this does not exclude bound water such as residual moisture or crystal water.

In one aspect of the present invention, composition C may be a solid composition at 25° C. and atmospheric pressure, preferably a powder composition or a pellet composition or a tablet composition or a capsule composition, more preferably a hair dyeing powder composition or a hair dyeing pellet composition or a hair dyeing tablet composition, still more preferably it is a hair dyeing powder composition.

The term ‘powder’ denotes a solid composition at 25° C. and atmospheric pressure. The term relates to freely flowing powders as well as compressed powders such as tablets. The powder composition may also comprise water as long as its nature of the solid state at 25° C. is unchanged. Depending on the type of powder, a water content of 10% by weight or less, calculated to the total weight of composition C, may be acceptable. This does not exclude the presence of residual moisture from air or crystal water bound to ingredients. Preferably, composition C is an anhydrous powder composition, from the viewpoint of stability.

Suitably, compositions C is an anhydrous powder composition, preferably an anhydrous hair dyeing powder composition.

### Lipophilic Compounds as Compounds According to Group d)

Compositions C and/or D may comprise one or more lipophilic compound(s) as compound(s) according to group d).

Preferably, compounds according to group d) are selected from C₁₂ to C₂₂ fatty alcohols, esters of C₃ to C₂₂ alcohols with C₁₂ to C₂₂ fatty acids, C₈ to C₂₂ fatty acids, vegetable oils, and/or silicones, and/or hydrocarbon-based products, and/or their mixtures, from the viewpoint of cosmetic compatibility.

Suitable C₁₂ to C₂₂ fatty alcohols are are myristyl alcohol, cetyl alcohol, stearyl alcohol, behenyl alcohol, and cetearyl alcohol.

Suitable esters of C₃ to C₂₂ alcohols with C₁₂ to C₂₂ fatty acids are isopropyl myristate, isopropyl palmitate, and myristyl myristate.

Suitable C₈ to C₂₂ fatty acids are oleic acid, linoleic acid, and palmitic acid.

Suitable vegetable oils are olive oil, almond oil, sunflower oil, and argan oil.

Suitable silicones are non-aminated and/or aminated silicones. The latter are commonly known as amodimethicones.

It is preferred from the viewpoint of forming a stable composition and user friendliness that the total concentration of compounds according to group d) is 1% by weight or more, more preferably 2% by weight or more, further more preferably 3% by weight or more, calculated to the total weight of each of the compositions C and/or D.

It is preferred from the viewpoint of forming a stable composition that the total concentration of compounds according to group d) is 20% by weight or less, more preferably 15% by weight or less, further more preferably 12% by weight or less, calculated to the total weight of each of the compositions C and/or D.

For attaining the above-mentioned effects, the total concentration of compounds according to group d) is in the range of 1% to 20% by weight, preferably in the range of 2% to 15% by weight, more preferably in the range of 3% to 12% by weight, calculated to the total weight of each of the compositions C and/or D.

### Liquid Composition

In another aspect of the present invention, composition C may be a liquid composition at 25° C. and atmospheric pressure comprising one or more organic solvent(s) as compound(s) according to group e), calculated to the total weight of composition C. Preferably, composition C is anhydrous, from the viewpoint of dye stability.

The term ‘liquid’ denotes a physical state at 25° C. and atmospheric pressure, i.e., composition C is/are liquid at room temperature.

The term ‘anhydrous’ denotes a composition C, which is/are free of added water. This does not exclude the presence of residual moisture from air or crystal water bound to ingredients.

For this aspect of the present invention, composition C may comprise one or more organic solvent(s).

The organic solvent(s) may be selected to dissolve the dyes. Preferred solvents are mono-, di-, and trivalent alcohols and/or their mixtures.

Preferred mono-, di-, and trivalent alcohols from the viewpoint of cosmetic safety and dissolution capacity are ethanol, n-propanol, isopropanol, propylene glycol, ethylene glycol, benzyl alcohol, phenoxyethanol, and glycerol, and/or their mixtures.

It is further preferred from the viewpoint of solution stability that the total concentration of organic solvents is 75% by weight or more, more preferably 80% by weight or more, further more preferably 85% by weight or more, calculated to the total weight of composition C.

It is further preferred from the viewpoint of dyeing intensity that the total concentration of organic solvents is 98% by weight or less, more preferably 95% by weight or less, further more preferably 92% by weight or less, calculated to the total weight of composition C.

For attaining the above-mentioned effects, it is preferred that the total concentration of organic solvents is in the range of 75% to 98% by weight, more preferably 80% to 95% by weight, further more preferably in the range of 85% to 92% by weight, calculated to the total weight of composition C.

### Hair Dyeing Oil

In one aspect of the present invention, composition C is a hair dyeing oil(s).

Optionally, composition C is a paste-like composition, preferably having a viscosity in the range of 10,000 mPas to 500,000 mPas, more preferably in the range of 20,000 mPas to 400,000 mPas, still more preferably in the range of 30,000 mPas to 250,000 mPas, measured by cone-plate viscometry at 25° C. and atmospheric pressure, preferably with a Brookfield viscometer with spindle #4 at 10 rpm for 1 min at 25° C. and atmospheric conditions.

For this purpose, that the total concentration of one or more compound(s) according to group d) is 90% by weight or less, preferably in the range of 50% to 90% by weight, calculated to the total weight of composition C.

Preferably, the paste-like compositions C are anhydrous, from the viewpoint of dye stability.

### Composition D

Composition D comprises one or more alkylamine salt(s) or alkanolamine salt(s) is/are according to the following general structure

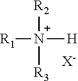

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and X— is an anion.

Preferably, from the viewpoint of solubility, the anion X— is sulfate, hydrochloride, chloride, hydrobromide, bromide, nitrate, phosphate, hydrogen phosphate, dihydrogen phosphate, silicate, disilicate, carbonate, bicarbonate, methosulfate, citrate, succinate, tartrate, lactate, malate, maleate, tosylate, benzoate, benzenesulfonate, acetate, and/or their mixtures, preferably the anion X— is sulfate.

It is further preferred from the viewpoint of commercial availability that one or more alkylamine salt(s) or alkanolamine salt(s) is/are salts of mono- and/or diethanolamine, butyl ethanolamine, butyl diethanolamine, dibutyl ethanolamine, methylethanolamine, triethanolamine, N-lauryl diethanolamine, diisopropanolamine, dimethyl isopropanolamine, isopropanolamine, triisopropanolamine, isobutanolamine, tris-(hydroxymethyl)-aminomethane, and/or their mixtures, more preferably it/they is/are mono- and/or diethanolamine salt(s), and/or their mixtures, more preferably they are monoethanolamine salt(s).

It is preferred from the viewpoint of wash fastness that the total concentration of compound(s) according to group b) is/are 0.1% by weight or more, more preferably 0.2% by weight or more, further more preferably 0.5% by weight or more, further more preferably 0.75% by weight or more, calculated to the total weight of composition A.

It is preferred from the viewpoint of cosmetic safety that the total concentration of compound(s) according to group b) is/are 30% by weight or less, more preferably 20% by weight or less, further more preferably 10% by weight or less, still more preferably 5% by weight or less, calculated to the total weight of composition A.

For attaining the above-mentioned effects, it is preferred that the total concentration of one or more compound(s) according to group b) is in the range 0.1% to 30% by weight, preferably in the range of 0.2% to 20% by weight, more preferably in the range of 0.5% to 10% by weight, still more preferably in the range of 0.75% to 5% by weight, calculated to the total weight of composition A.

It is further preferred from the viewpoint of wash fastness that the weight ratio of compound(s) according to group a) to compound(s) according to group b) in composition A is in the range of 10 to 0.001, preferably in the range of 8 to 0.005, more preferably in the range of 8 to 0.0075.

Composition D comprises hydrogen peroxide as compound according to g).

It is further preferred from the viewpoint of dyeing performance that the concentration of hydrogen peroxide in compositions D is 0.1% by weight or more, more preferably 0.25% by weight or more, further more preferably 1% by weight or more, calculated to the total weight of composition D.

It is further preferred from the viewpoint of product performance and user safety that the concentration of hydrogen peroxide in composition D is 20% by weight or less, more preferably 15% by weight or less, further more preferably 12% by weight or less, calculated to the total weight of composition D.

For attaining the above-mentioned effects, it is preferred that the concentration of hydrogen peroxide in composition D is in the range of 0.1% to 20% by weight, more preferably in the range of 0.25% to 15% by weight, further more preferably in the range of 1% to 12% by weight, calculated to the total weight of composition D.

It is preferred from the viewpoint of stability and dyeing performance that composition D has a pH in the range of 1.5 to 5, more preferably in the range of 2 to 4.5.

It is preferred from the viewpoint of stability that compositions C and D are kept separate until directly prior to use onto keratin fibers.

Suitable mixing ratios by weight of compositions C and D are in the range of 1:1 to 1:20, preferably in the range of 1:2 to 1:15, more preferably in the range of 1:4 to 1:12, still more preferably in the range of 1:5 to 1:10.

### Surfactants as Compounds According to f)

Compositions C and/or D may further comprise one or more surfactant(s) as compound according to group f), preferably selected from non-ionic surfactants, anionic surfactants, cationic surfactants, and/or amphoteric/zwitterionic surfactants, and/or their salt(s), and/or their mixtures, more preferably selected from anionic surfactants and/or their salt(s), from the viewpoint of stabilizing the composition and improving wettability and mixability.

Preferably, the anionic surfactants may be selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures, and/or their salts.

Suitable examples are alkyl sulfate or preferably ethoxylated alkyl ether sulfate surfactants or mixtures thereof, and/or salts thereof, having an alkyl chain length of C₁₀ to C₂₂ and an ethoxylation degree from 1 to 50.

Suitable non-ionic surfactants may be selected from alkyl polyglycosides, ethoxylated triglycerides, ethoxylated fatty alcohols, ethoxylated fatty acid esters, and/or their mixtures.

Suitable cationic surfactants are quaternary ammonium surfactants having a carbon chain length in the range of C₁₂ to C₂₂ or surfactants having a tertiary amine group and at least one alkyl chain having a carbon chain length in the range of C₁₂ to C₂₂ such as alkylamidoalkylamine surfactants, and/or their salts. Suitable examples are cetrimonium chloride and behentrimonium chloride.

Suitable amphoteric/zwitterionic surfactants are of betaine type. Suitable compounds may be selected from alkyl betaines and/or alkylamido betaines. A preferred compound selected from alkyl betaines is lauryl betaine. A preferred compound selected from alkylamido betaines is cocamidopropyl betaine. The disclosure also relates to the salts of the compounds.

Suitable concentration ranges for surfactants are in the range of 0.1% to 10% by weight, calculated to the total weight of each of the compositions C and/or D, from the viewpoint of enhancing wettability of keratin fibers, physical stability, and mixability with other compositions.

### Thickening Polymers

From the viewpoint of cosmetic safety, it is further preferred that the compositions C and/or D comprise one or more thickening polymer.

Composition C and/or D may comprise one or more thickening polymer(s) selected from non-ionic thickening polymers and/or anionic thickening polymers, and/or their mixtures.

Preferably, the thickening polymers are selected from polymers resulting in an aqueous solution and/or aqueous dispersion at pH ranges between 7 and 12 having a viscosity of at least 1,000 mPa·s measured at a polymer concentration of 1% by weight in water at 25° C. determined by cone plate viscometry at 25° C. under atmospheric conditions, calculated to the total weight of the composition, determined by a Brookfield viscometer, such as at 10 rpm for 1 min, with an appropriate spindle at 25° C.

Suitable non-ionic thickening polymers are cellulose-based polymers. Suitable examples of cellulose-based polymers are methylcellulose, hydroxymethylcellulose, hydroxyethylcellulose, hydroxypropylcellulose, hydroxyethyl-methylcellulose, and alkylated hydroxyl celluloses such as (C₂-C₈)-alkylcelluloses or cetyl hydroxyethylcellulose.

Suitable anionic thickening polymers are selected from naturally-based anionic polymers and/or synthetic anionic polymers.

Suitably, the natural anionic polymer(s) may be selected from xanthan gum, dehydroxanthan gum, hydroxypropylxanthan gum, carboxymethyl cellulose and starch based polymers such as vegetable starch and/or their synthetically modified derivatives such as hydroxypropyl starch phosphate. Equally suitable are alginic acids, sodium alginates, ammonium alginates, calcium alginates, gum arabic, and guar gum.

Suitable synthetic anionic polymers are associative thickening polymers, such as acrylates/steareth-30 methacrylate copolymer.

The preferred thickening polymer for the composition of the present invention are natural anionic polymers, more preferably xanthan gum and/or dehydroxanthan gum, from the viewpoint of their biodegradability and low environmental impact.

Preferably, the total concentration of thickening polymers of the present invention are 0.1% by weight or more, more preferably 0.25% by weight or more, more preferably 0.5% by weight or more, calculated to the total weight of each of the compositions C and/or D, from the viewpoint of providing sufficient viscosity to the composition.

Preferably, the total concentration of thickening polymers of the present invention are 15% by weight or less, more preferably 12% by weight or more, further more preferably 10% by weight or less, calculated to the total weight of each of the compositions C and/or D, from the viewpoint of providing sufficient viscosity to the composition and cost of goods.

For attaining the above-mentioned effects, it is preferred that the total concentration of thickening polymers in the composition of the present invention is in the range of 0.1% to 15% by weight, preferably in the range of 0.25% to 12% by weight, more preferably in the range of 0.5% to 10% by weight, calculated to the total weight of each of the compositions C and/or D.

It is preferred from the viewpoint of cosmetic safety that the compositions C and/or D, in case they are aqueous, have a viscosity in the range of 1,000 Pas to 25,000 mPas, preferably 2,000 mPas to 20,000 mPas, more preferably in the range of 2,500 mPas to 17,500 mPas, determined by cone plate viscometry at 25° C. under atmospheric conditions, preferably with a Brookfield viscometer with spindle #4 at 10 rpm for 1 min at 25° C. and atmospheric pressure.

### Method for Dyeing

The present invention is also directed to a method for dyeing keratin fibers, preferably human keratin fibers, more preferably human hair, comprising the steps of:
x) providing composition C as defined above and mixing it with a composition D as defined above, to yield a ready-to-use composition having a pH in the range of 7 to 12,
xi) applying the ready-to-use composition onto keratin fibers and leaving it for a time period in the range of 1 min to 60 min,
xii) optionally rinsing-off the keratin fibers and optionally drying the keratin fibers.

If composition C comprises oxidizing agents, then composition D may be free of oxidizing agents.

The ready-to-use composition is applied to keratin fibers and preferably left for a time period of 1 min to 60 min as defined in step xi). Further preferred time ranges for step xi) are 5 min to 45 min, more preferred ranges are 10 min to 35 min, from the viewpoint of sufficiently dyeing.

Optionally, heat may be applied while leaving the composition C or the ready-to-use composition onto keratin fibers. Suitable temperature ranges are 30° C. to 50° C.

Preferably, from the viewpoint of saving resources, the mixing ratio by weight of compositions C and D in step x) is in the range of 1:1 to 1:20, preferably in the range of 1:2 to 1:15, more preferably in the range of 1:4 to 1:12, still more preferably in the range of 1:5 to 1:10.

The following examples are to illustrate the invention, but not to limit it.

### EXAMPLES

TABLE 1
Compositions C

| | Inv. | Inv. | Inv. | Comp. | Comp. | Comp. |
|---|---|---|---|---|---|---|
| | Ex. 1 | Ex. 2 | Ex. 3 | Ex. 1 | Ex. 2 | Ex. 3 |
| Ingredients | [% by weight] | | | | | |
| 1,4-diamino-2- | 5.0 | 5.0 | 5.0 | — | — | — |
| methoxymethyl- | | | | | | |
| benzene | | | | | | |
| p-toluene | — | — | — | 5.0 | 5.0 | 5.0 |
| diamine sulfate | | | | | | |
| Resorcinol | 5.0 | — | — | 5.0 | — | — |
| 2-Methylresorcinol | — | 5.0 | — | — | 5.0 | — |
| 2-Amino-3- | — | — | 5.0 | — | — | 5.0 |
| hydroxypyridine | | | | | | |

| | |
|---|---|
| Sodium sulfite | 0.5 |
| Ammonium hydroxide | 8.8 |
| Ascorbic acid | 0.2 |
| Water | Ad 100.0 |

Wash fastness

| | | | | | | |
|---|---|---|---|---|---|---|
| Upon 20x washes | 0.56 | 0.69 | 3.47 | 2.52 | 1.04 | 5.18 |
| (ΔΔE) | | | | | | |

The compositions of table 1 had a pH in the range of 10.1±0.3.

### Aqueous Composition D

% by weight
Monoethanolamine sulfate 7.0
Sulfuric acid 0.3
Hydrogen peroxide 4.5
Hydroxy-4-acetanilid 0.015
Water ad 100.0

The inventive compositions showed better wash fastness in comparison to the comparative compositions.

TABLE 2

| | | Inv. | Inv. | Inv. | Comp. |
|---|---|---|---|---|---|
| | | Ex. 4 | Ex. 5 | Ex. 6 | Ex. 4 |
| Ingredients | | [% by weight] | | | |
| Composition | 1,4-diamino-2- | 0.2 | 0.2 | 0.2 | 0.2 |
| C | methoxymethyl- | | | | |
| | benzene | | | | |
| | Resorcinol | 0.1448 | 0.1448 | 0.1448 | 0.1448 |
| | Ammonium | 0.88 | 0.88 | 0.88 | 0.88 |
| | hydroxide | | | | |
| | Monoethanolamine | — | — | — | 4.4 |
| | Sodium sulfite | 0.05 | 0.05 | 0.05 | 0.05 |
| | Ascorbic acid | 0.05 | 0.05 | 0.05 | 0.05 |
| | Water | Ad 100.0 | | | |
| Composition | Monoethanolamine | 2.0 | 15.0 | | — |
| D | sulfate | | | | |
| | Monoethanolamine | — | | 7.0 | — |
| | hydrochloride | | | | |
| | Sulfuric acid (10% | 0.3 | 0.3 | 0.3 | 0.3 |
| | active) | | | | |
| | Hydrogen peroxide | 9.0 | 9.0 | 9.0 | 9.0 |
| | (50% active) | | | | |
| | Water | Ad 100.0 | | | |
| Evaluation | ΔE upon dyeing | 64.9 | 66.2 | 65.4 | 59.8 |
| | ΔE upon washing | 62.9 | 65.8 | 63.6 | 56.1 |
| | ΔΔE | 2.0 | 0.4 | 1.8 | 3.7 |

The ready-to-use compositions prepared by the mixture of compositions C and D (weight ratio 1:1) of table 2 directly prior to application onto hair had a pH in the range of 10.0±0.5. MEA HCl correspond to MEA in molar concentrations in inventive ex. 6 and comp. ex. 4.

The inventive compositions showed better dyeing intensity and better wash fastness in comparison to the comparative composition.

### Methods

### Hair Dyeing

Goat hairstreaks (21 cm, 2 g per bundle) were permed with 2 g of a commercial perming product available under the trade name Goldwell Topform Type 1 for 20 min, rinsed, and then treated with an oxidative composition for 10 min. The hair streaks were shampooed and dried.

The dyeing compositions of tables 1 were mixed with the aqueous composition B in a weight ratio of 1:1 to prepare ready-to-use compositions having a pH in the range of 9.5 to 10.0.

2 g of the ready-to-use compositions were applied onto the permed goat hairstreaks for 30 min at 40° C. The streaks were then rinsed off and dried. The colormetric data were obtained with a color-difference meter (Datacolor 600) in the CIE colorimetric system (L*, a*, b*). Color differences (ΔE) were calculated by the following formula.

$$Δ E = \sqrt{{( {L}_{1}^{*} - {L}_{0}^{*} )}^{2} + {( {a}_{1}^{*} - {a}_{0}^{*} )}^{2} + {( {b}_{1}^{*} - {b}_{0}^{*} )}^{2}}$$

### Wash Fastness

The dyed hair streaks were placed in a water bath comprising 3.5% by weight of sodium laureth sulfate and shaking it for 30 min at 40° C. This step was repeated for 20 times with a drying step after every 5 washes. After 20 times shaking bath treatment, the hairstreaks were blow-dried and the remaining color was measured as described above. The color differences between 20 times washed and freshly colored hair were reported as ΔΔE.

The following examples are within the scope of the present invention.

Inventive Example 7
Composition C

| | % by weight |
|---|---|
| 1,4-diamino-2-methoxymethyl-benzene | 0.097 |
| 5-Amino-6-chloro-o-cresol | 0.1 |
| HC Blue 18 | 0.05 |
| HC Red 18 | 0.08 |
| HC Yellow 16 | 0.02 |
| Monoethanolamine sulfate | 10.0 |
| Diatomaceous earth | ad 100.0 |

Composition C is mixed with the aqueous composition D of the examples from table 1 in a weight ratio of 1:1 to prepare ready-to-use compositions having a pH in the range of 9.5 to 10.0.

Inventive Example 8
Composition C

| | % by weight |
|---|---|
| 1,4-diamino-2-methoxymethyl-benzene sulfate | 0.097 |
| 5-Amino-6-chloro-o-cresol | 0.1 |
| HC Blue 18 | 0.05 |
| HC Red 18 | 0.08 |
| HC Yellow 16 | 0.02 |
| 2-amino-2-methylpropanol hydrochloride | 10.0 |
| 1,2-propylene glycol | ad 100.0 |

Composition C is mixed with the aqueous composition D of the examples from table 1 in a weight ratio of 1:1 to prepare ready-to-use compositions having a pH in the range of 9.5 to 10.0.

1. A dyeing product for keratin fibers, comprising:
a composition C, comprising:
a) 1,4-diamino-2-methoxymethyl-benzene, and/or a salt thereof,
an aqueous composition D having a pH in a range of 1 to 6 comprising:
b) an alkylamine salt or alkanolamine salt according to the following general structure 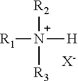
wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and X— is an anion, and
g) hydrogen peroxide,
wherein the composition C and the aqueous composition D are kept separate until directly prior to use onto the keratin fibers.

2. The dyeing product according to claim 1, wherein a total concentration of the 1,4-diamino-2-methoxymethyl-benzene in the composition C is in a range of 0.01% to 65% by weight, calculated to a total weight of the composition C.

3. The dyeing product according to claim 1, wherein the composition C further comprises an oxidative dye precursor or oxidative dye coupler, different from the 1,4-diamino-2-methoxymethyl-benzene, which is at least one selected from 5-amino-2-methylphenol, 2-methyl-5-hydroxyethylaminophenol, 2,4,-diaminophenoxyethanol, 2-amino-4-hydroxyethylaminoanisol, 2-methyl-5-amino-6-chlorphenol, 1,3-bis-(2,4-diaminophenoxy)-propane, 2-bis(2-hydroxyethyl)-aminotoluene, 2-amino-5-methylphenol, resorcinol, 2-methylresorcinol, 4-chlororesorcinol, 2-amino-4-chlorophenol, 5-amino-4-methoxy-2-methylphenol, 2-aminophenol, 3-amino-phenol, 1-methyl-2-hydroxy-4-aminobenzene, 3-N,N-dimethyl aminophenol, 2,6-dihydroxy-3,5-dimethoxypyridine, 5-amino-3-methylphenol, 6-amino-3-methylphenol, 2,4-diaminophenoxyethanol, 1,3-diamino-benzene, 1-amino-3-(2′-hy-droxyethylamino) benzene, 1-amino-3-[bis(2′-hydroxy-ethyl)amino]benzene, α-naphthol, 4,6-dichlororesorcinol, 1,3-diamino-toluene, 4-hydroxy-1,2-methylenedioxy benzene, 1,5-dihydroxy naphthalene, 1,6-dihydroxy naphthalene, 1,7-dihydroxy naphthalene, 2,7-dihydroxy naphthalene, 1-hydroxy-2-methyl naphthalene, 4-hydroxy-1,2-methyldioxy benzene, 2,4-diamino-3-chlorophenol, 5-amino-2-methoxyphenol and/or 1-methoxy-2-amino-4-(2′-hydroxyethyl amino)-benzene, 2,4,5,6-tetraaminopyrimidine, 2,5,6-triamino-5-pyrimidinol, 2-amino-3-hydroxypyridine, 5-amino-6-chloro-o-cresol, 3-amino-2,4-dichlorophenol, 1,3-bis-(2,4-diamino-phenoxy)-propane, 2-amino-4-hydroxyethylaminoanisole, salts thereof, or mixtures thereof.

4. The dyeing product according to claim 3, wherein a total concentration of the oxidative dye precursor or oxidative dye coupler different from the 1,4-diamino-2-methoxymethyl-benzene in the composition C is in a range of 0.01% to 30% by weight, calculated to a total weight of the composition C.

5. The dyeing product according to claim 1, wherein the composition C further comprises an alkalizing agent which is at least one selected from metasilicates, disilicates, carbonates, bicarbonates, phosphates, and/or alkali or earth alkaline salts thereof, and/or alkyl or alkanolamines, or mixtures thereof.

6-8 (canceled)

9. The dyeing product according to claim 5, wherein a total concentration of the alkalizing agent in the composition C is in a range of 0.1% to 65% by weight, calculated to a total weight of the composition C.

10. The dyeing product according to claim 1, wherein the composition C further comprises a direct dye which is at least one selected from HC Blue 18, HC Red 18, HC Yellow 16, Disperse Black 9, Acid Yellow 1, 2-amino-6-chloro-4-nitrophenol, salts thereof, or mixtures thereof.

11. (canceled)

12. The dyeing product according to claim 1, wherein the composition C further comprises a pulverulent excipient which is at least one selected from diatomaceous earth, kaolin, bentonite, starch, nylon powder, montmorillonit, gypsum, sawdust, perlite or mixtures thereof.

13. (canceled)

14. The dyeing product according to claim 1, wherein the composition C is a solid composition at 25° C. and atmospheric pressure.

15. (canceled)

16. The dyeing product according to claim 1, wherein a total concentration of the alkylamine salt or alkanolamine salt in the aqueous composition D is in a range of 0.1% to 30% by weight, calculated to a total weight of the aqueous composition D.

17. The dyeing product according to claim 1, wherein the composition C and/or the aqueous composition D further comprise a lipophilic compound which is at least one selected from C₁₂ to C₂₂ fatty alcohols, esters of C₃ to C₂₂ alcohols with C₁₂ to C₂₂ fatty acids, C₈ to C₂₂ fatty acids, vegetable oils, silicones, hydrocarbon-based products or mixtures thereof.

18-19. (canceled)

20. The dyeing product according to claim 1, wherein the composition C is a hair dyeing oil.

21-25. (canceled)

26. The dyeing product according to claim 1, wherein the composition C and/or the aqueous composition D further comprise a thickening polymer which is at least one selected from non-ionic thickening polymers, anionic thickening polymers, or mixtures thereof.

27-30. (canceled)

31. The dyeing product according to claim 1, wherein the aqueous composition D has a pH in a range of 2 to 4.5.

32. The dyeing product according to claim 1, wherein a total concentration of the hydrogen peroxide in the aqueous composition D is in a range of 0.1% to 20% by weight, calculated to a total weight of the aqueous composition D.

33. The dyeing product according to claim 1, wherein a mixing ratio by weight of the composition C to the aqueous composition D is in a range of 1:1 to 1:20.

34. A method for dyeing keratin fibers with the dyeing product of claim 1, comprising:
mixing the composition C and the aqueous composition D, to yield a ready-to-use composition having a pH in a range of 7 to 12;
applying the ready-to-use composition onto the keratin fibers and leaving it for a time period in a range of 1 min to 60 min; and
optionally rinsing-off the keratin fibers and optionally drying the keratin fibers.

35-36. (canceled)

37. The method according to claim 34, wherein a mixing ratio by weight of the composition C to the aqueous composition D is in a range of 1:1 to 1:20.

38. The dyeing product according to claim 5, wherein the alkalizing agent is at least one selected from sodium metasilicate, trisodium phosphate, tripotassium phosphate, tris-(hydroxymethyl)-aminomethane, or mixtures thereof.

39. The dyeing product according to claim 1, wherein the alkylamine salt or alkanolamine salt of the aqueous composition D is at least one selected from salts of mono- and/or diethanolamine, butyl ethanolamine, butyl diethanolamine, dibutyl ethanolamine, methylethanolamine, triethanolamine, N-lauryl diethanolamine, diisopropanolamine, dimethyl isopropanolamine, isopropanolamine, triisopropanolamine, isobutanolamine, tris-(hydroxymethyl)-aminomethane, or mixtures thereof.
